(19) United States
(12) Patent Application Publication
(10) Pub. No.: US 2024/0299699 A1
(43) Pub. Date: Sep. 12, 2024

(54) PERSONALIZED INFANT SLEEP AID WITH AI-BASED GRADUAL ADJUSTMENT

(71) Applicant: Ryan Redford, Irvine, CA (US)

(72) Inventor: Ryan Redford, Irvine, CA (US)

(21) Appl. No.: 18/601,631

(22) Filed: Mar. 11, 2024

Related U.S. Application Data
(60) Provisional application No. 63/451,482, filed on Mar. 10, 2023.

(51) Int. Cl. A61M 21/02; A61M 21/00; G06F 3/16; G10L 25/51; G10L 25/90; G16H 40/67

(52) U.S. Cl. CPC A61M 21/02; G06F 3/165; G10L 25/51; G10L 25/90; G16H 40/67; A61M 2021/0027; A61M 2205/3375; A61M 2240/00

(57) ABSTRACT

A device for promoting healthy sleep habits in an individual, including a recording device for capturing the voices of the individual's parent(s) or care giver, a processing unit for analyzing the recorded voices and creating a customized soothing method for the individual, and a speaker for playing the customized soothing method. The device also includes a mechanism for gradually adjusting the soothing method over time to train the individual for healthy sleep practices.

### CROSS REFERENCE TO RELATED APPLICATION

This application claims priority from U.S. Provisional Application Ser. No. 63/451,482, filed Mar. 10, 2023, the contents of which are incorporated herein in their entirety.

### BACKGROUND AND SUMMARY

There is a growing body of research on the importance of healthy sleep habits for newborns, and the potential benefits of using customized soothing methods to promote these habits. Studies have shown that infants who are able to establish healthy sleep patterns early in life tend to have better overall health and development outcomes, including improved cognitive development, stronger immune systems, and reduced risk of obesity and other chronic health conditions.

Some studies have specifically looked at the use of customized sounds and white noise in promoting healthy sleep habits in infants. For example, a study published in the

Journal of Sleep Research found that infants exposed to a customized white noise intervention fell asleep faster and had longer periods of uninterrupted sleep compared to a control group. Another study, published in the Journal of Developmental and Behavioral

Pediatrics, found that a customized lullaby intervention led to improved sleep patterns in infants.

Additionally, studies have shown that Artificial Intelligence (AI)-based systems for analyzing and creating personalized soundscapes for infants can be effective. A study published in the International Journal of Human-Computer Studies found that an AI-based system for creating personalized lullabies resulted in improved sleep outcomes for infants compared to a control group.

Thus, these studies suggest that utilizing AI-based systems to create customized soothing methods for infants, tailored to the individual child, can be an effective way to promote healthy sleep habits.

The present disclosure provides a device for promoting healthy sleep habits in newborns. The device comprises a recording device configured for capturing the voices of the child's parents or care giver, a processing unit configured for analyzing the recorded voices and creating a customized soothing method for the child, and a speaker configured for playing the customized soothing method. The device also includes a processor or processing unit configured to gradually adjust the soothing method over time to train the child for healthy sleep practices.

The recording device may be a microphone or other device configured for capturing audio input. The processing unit may be a computer or other device configured for analyzing audio input and creating a customized soothing method. The speaker may be any device configured for playing audio output, such as audio speakers or a headphone.

The processing unit is configured to utilize Artificial Intelligence (AI) algorithms to analyze the recorded voices of the parents and identify certain characteristics of the recorded voices, such as pitch, rhythm, and tone, and to create a soothing method that incorporates these characteristics and is tailored to the individual child. The soothing method may include white noise, nature sounds, or other calming sounds including recorded sounds of the parent voices.

The device also includes a controller for gradually adjusting the soothing method over time to train the child for healthy sleep practices. For example, the device may be configured to start by playing the soothing method for a longer period of time and gradually decrease the time over a period of weeks or months.

In one embodiment, the device also may include a timer configured for controlling the duration of the soothing method, and a volume control configured for adjusting the volume of the audio output.

In another embodiment, the device may include a remote control configured to permit adjustment of settings and for controlling the device remotely.

In yet another embodiment, the device may include a battery or other portable power source, and a connector for connecting to a power source for charging.

The present disclosure also provides a device configured to customize a soothing method to the individual child, and gradually adjust the soothing method over time to train the individual child for healthy sleep practices. This can lead to better sleep habits in newborns and can ultimately lead to better overall health and development for the child.

In yet another embodiment, the device provides capturing the voices of the individual's or child's parents using a recording device, analyzing the recorded voices using a processing unit to create a customized soothing method for the individual child, playing the customized soothing method using a speaker and gradually adjusting the soothing method over time to train the child or individual for healthy sleep practices.

### BRIEF DESCRIPTION OF THE DRAWINGS

Further features and advantages of the instant disclosure will be seen from the following detailed description taken in conjunction with the accompanying drawings, wherein like numerals depict like parts, and wherein:

FIG. 1 is a schematic drawing of a device for promoting healthy sleep habits in individual children and newborns.

### DETAILED DESCRIPTION

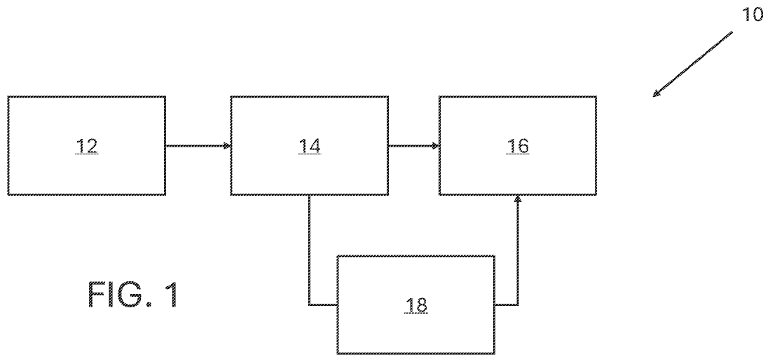

Referring to FIG. 1, a device 10 for promoting healthy sleep patterns in individual child or newborns includes a recording device 12 configured for capturing voices of a individual child's parents, processing unit 14 configured for analyzing the recorded voices and creating a customized soothing method for the child, and a speaker 16 configured for playing the customized soothing method. The device also includes a controller 18 configured to gradually adjust the soothing method over time to train the child for health sleep practices.

The processing unit 14 is configured to create a soothing method tailored to the individual child. As noted supra, the soothing method may comprise white noise, nature sounds and other calming sounds including recording sounds of the parent voices. The controller 18 uses mathematical equations and algorithms that are used with Artificial Intelligence (AI) to analyze recorded voices of the parents and identify certain characteristics such as pitch, rhythm and tone, and then creates a soothing method to incorporate these characteristics that is tailored to the individual child.

There are a variety of mathematical equations and algorithms that can be used for AI implementations, depending on the specific task and application. Mathematical equations and algorithms in AI useful in practicing the subject disclosure include:
1. Linear Regression: This is a statistical method that is used to model the relationship between a dependent variable and one or more independent variables. Linear regression can be used for tasks such as predicting numerical values or classifying data.
2. Logistic Regression: This is a statistical method that is used for binary classification tasks, where the goal is to predict one of two possible outcomes. Logistic regression can be used for tasks such as determining whether an email is spam or not.
3. Decision Trees: This is a method for creating a model that can be used to make decisions based on a set of input features. Decision trees can be used for tasks such as classifying data or making predictions.
4. Random Forest: This is an ensemble method that combines multiple decision trees to create a more robust and accurate model. Random forest can be used for tasks such as image recognition or natural language processing.
5. Neural Networks: This is a method that is based on the structure and function of the human brain and can be used for tasks such as image recognition, natural language processing, and speech recognition.
6. Convolutional Neural Networks (CNN): This is a type of neural network that is commonly used for image recognition and other computer vision tasks. CNNs are designed to process data with grid-like topology, such as an image.
7. Recurrent Neural Networks (RNN): This is a type of neural network that is used for tasks that involve sequential data, such as natural language processing and speech recognition. RNNs are designed to process sequences of data, where the order of the data is important.
8. Long short-term memory (LSTM): This is a variation of RNN that is able to handle long-term dependencies and is used in tasks such as speech recognition, natural language processing, and time-series prediction.
9. Support Vector Machines (SVM): This is a method used for classification tasks, it separates data points of different classes by finding the best hyperplane that maximizes the margin between them.

### Summarizing to This Point

The present disclosure uses the voices of the individual child's parents to create a customized soothing method for the child, which is tailored to the individual child's needs and preferences. This is far superior to conventional sleep-promoting devices that use pre-recorded sounds or white noise, as the device creates a personalized soothing method for the child.
The device gradually adjusts the soothing method over time to train the individual child for healthy sleep practices. This also is far superior to conventional sleep-promoting devices that use a fixed soothing method, which may not be effective over time as the child's needs change.
The device uses AI algorithms to analyze the recorded voices of the parents and identify certain characteristics, such as pitch, rhythm, and tone. This also is far superior to conventional sleep-promoting devices that do not use AI-based analysis.
The device has many features such as volume control, timer, remote control, etc. which are not commonly found in conventional sleep-promoting devices.

While the foregoing disclosure has been directed to providing personalized infant sleep aid, the disclosure also advantageously may be used with older children who have sleep issues, as well as individuals of any age who have sleep issues due to anxiety or other causes that can be smoothed over by comforting sounds of voices of a parent or caregiver.

1. A device for promoting healthy sleep habits, comprising: a recording device configured for capturing the voices of an individual child's parent(s) or care giver; a processing unit configured for analyzing the recorded voices and creating a customized soothing method for the individual; a speaker configured for playing the customized soothing method; and a processing unit for gradually adjusting the soothing method over time to train the individual for healthy sleep practices.

2. The device of claim 1, wherein the recording device is a device configured for capturing audio input.

3. The device of claim 2, wherein the recording device is a microphone.

4. The device of claim 1, wherein the processing unit is a computer configured to analyze audio input and to create a customized soothing method.

5. The device of claim 1, wherein the speaker is a device configured to play audio output.

6. The device of claim 5, wherein the speaker is an audio speaker or a headphone.

7. The device of claim 1, wherein the processing unit is configured to utilize Artificial Intelligence (AI) algorithms configured to analyze the recorded voices of the parents and identify characteristics of the recorded voices.

8. The device of claim 7, wherein the characteristics are selected from the group consisting of pitch, rhythm and tone.

9. The device of claim 1, further comprising a timer configured for controlling the duration of the soothing method.

10. The device of claim 1, further comprising a volume control configured for adjusting the volume of the audio output.

11. The device of claim 1, further comprising a remote control configured for permitting remote adjustment of the settings and permitting controlling the device remotely.

12. The device of claim 1, further comprising a battery or other portable power source, and a connector for connecting to a power source for charging.

13. A method for promoting healthy sleep habits comprising the steps of: a) capturing the voices of the child's parents using a recording device; b) analyzing the recorded voices using a processing unit to create a customized soothing method for the child; c) playing the customized soothing method using a speaker; and d) gradually adjusting the soothing method over time to train the child for healthy sleep practices.
